(19) United States
(12) Patent Application Publication
(10) Pub. No.: US 2019/0142646 A1
(43) Pub. Date: May 16, 2019

(54) MODIFIABLE BANDAGE SYSTEM AND METHOD

(71) Applicants: David Halvorson, Everett, WA (US); David Pitkethly, Everett, WA (US)

(72) Inventors: David Halvorson, Everett, WA (US); David Pitkethly, Everett, WA (US)

(21) Appl. No.: 16/192,716

(22) Filed: Nov. 15, 2018

Related U.S. Application Data
(60) Provisional application No. 62/586,742, filed on Nov. 15, 2017.

(51) Int. Cl. A61F 13/02; A61F 13/00; A61F 15/02; A61B 17/06

(52) U.S. Cl. CPC A61F 13/0206; A61F 13/00076; A61F 13/0266; A61F 15/02; A61F 2013/00119; A61F 13/0253; A61F 13/00063; A61F 13/00059; A61B 17/06114

(57) ABSTRACT

A modifiable bandage system; the modifiable bandage system is a modifiable bandage that includes a malleable material, an attached absorbent material, and a removable backing. The malleable material includes a longitudinal severable indicia that defines a severable-longitudinal area. The malleable material further includes a transverse severable indicia that defines a severable-transverse area. The modifiable bandage system functions such that absorbent material may be extended longitudinally by removal of the severable-transverse area and coupling multiple modifiable bandages. Further, the absorbent material may be extended transversely by removal of the severable-longitudinal area and coupling multiple modifiable bandages. The modifiable bandage system is useful for covering various sizes of wounds.

### CROSS-REFERENCE TO RELATED APPLICATION(S)

The present application is related to and claims priority to U.S. Provisional Patent Application No. 62/586,742 filed Nov. 15, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of wound dressing of existing art and more specifically relates to bandages.

### RELATED ART

Wound dressings are common to every human who endures a cut or tear on their skin. An epidermis is the outer layer of cells that cover and protect the inner dermis. The dermis is comprised of a tick layer of living tissue that contains blood capillaries, nerve endings, sweat glands, hair follicles, and other internal structures. The dermis must be protected from foreign materials and microbes that may cause infection if introduced to the bloodstream. A laceration is a cut or tear in the epidermis, vasculature, and commonly the dermis and underlying tissues depending on the depth of the wound. A laceration may provide access for harmful foreign matter to infect the body. A laceration is typically tended to by a material that blocks the new port of access for the potentially life-threatening invading microbes. Historically, lacerations were tended to using trial-and-error with various substances such as herbs, honey, wine, vinegars, and anything found to have anti-bacterial properties. A wool boiled in water or wine was considered an appropriate dressing for a wound. Presently, mankind has developed more advanced methods of treating wounds. A commonly used bandage is a unitary implement having an absorbent material such as gauze that is applied directly to a laceration. The commonly used bandage has a very limited size restriction for the coverage area. Alternative methods of treating a wound that is outside of the size restriction of the commonly used bandage may include multiple materials that must be size customized for the wound. These methods that use multiple materials may be cost prohibitive, or the materials not readily available when needed. A suitable solution is desired.

U.S. Pat. No. 5,947,917 to Theresa Carte relates to an adhesive bandage or tape. The described adhesive bandage or tape includes a water resistant, flexible, dermal adhesive product for conformable topical application to human skin, the dermal adhesive product comprising a backing sheet having an adhesive for removably adhering the sheet to the skin, wherein the adhesive is a highly crosslinked pressure sensitive adhesive composition comprising the polymerization reaction product of about 75% to about 95% of a mixture of at least two alkyl acrylate or methacrylate esters, about 1% to about 10% ethylenically unsaturated carboxylic acid, and about 10% to about 20% vinyl lactam, by dry weight of solids, preferably about 80% to about 90% of a mixture of at least two alkyl acrylate or methacrylate esters, about 2% to about 5% ethylenically unsaturated carboxylic acid, and about 10% to about 20% vinyl lactam, by dry weight of solids.

### SUMMARY OF THE INVENTION

In view of the foregoing disadvantages inherent in the known wound dressing art, the present disclosure provides a novel modifiable bandage system and method. The general purpose of the present disclosure, which will be described subsequently in greater detail, is to provide an efficient and effective modifiable bandage system and method.

A modifiable bandage system is disclosed herein. Briefly stated, the modifiable bandage system includes a modifiable bandage including a malleable material, an attached absorbent material, and a removable backing. The malleable material includes a longitudinal severable indicia that defines a severable-longitudinal area. The malleable material further includes a transverse severable indicia that defines a severable-transverse area. The modifiable bandage system functions such that absorbent material may be extended longitudinally by removal of the severable-transverse area and coupling multiple modifiable bandages. Further, the absorbent material may be extended transversely by removal of the severable-longitudinal area and coupling multiple modifiable bandages.

More specifically, the modifiable bandage includes a substantially rectangular malleable material having opposing long-edges and opposing short-edges. The malleable material has a top-surface that includes a substantially fluid-proof laminate and, a bottom-surface that includes an adhesive material. The absorbent material is disposed on the bottom-surface of the malleable material. The absorbent material extends longitudinally a first-distance from each of the short-edges of the malleable material. The absorbent material also extends transversely a second-distance from each of the long-edges of the malleable material. The absorbent material is disposed on the bottom-surface of the malleable material such that the absorbent-material is circumscribed by the adhesive material. The longitudinal severable indicia is disposed on the top-surface of the malleable material. The longitudinal severable indicia is a linear marker that extends between the opposing short-edges of the malleable material. The longitudinal severable indicia is disposed the aforementioned second-distance away from at least one of the long-edges. The longitudinal severable indicia defines a severable-longitudinal area. Similarly, the transverse severable indicia is a linear marker that is disposed on the top-surface of the malleable material. The transverse severable indicia extends between opposing long-edges the aforementioned first-distance away from at least one of the short-edges. The transverse severable indicia defines a severable-transverse area. The removable backing is simply removably attached to the bottom-surface of the malleable material and serves the function of covering the adhesive material.

According to another embodiment, a method of dressing a wound with a modifiable bandage system is also disclosed herein. The method of dressing a wound with a modifiable bandage system includes a step of providing a modifiable bandage system as described above. A next step comprises, removing the removable backing from the bottom-surface of the malleable material; next a step of, aligning the absorbent material over the wound; further a step of, pressing the modifiable bandage onto the wound such that the adhesive material circumscribes the wound and absorbent material covers the wound. The method further includes the optional steps as listed hereafter: a step of separating the severable-longitudinal area from the modifiable bandage; a step of coupling plural of modifiable bandage transversely; and a step of separating the severable-transverse area from the modifiable bandage. Lastly, is a step of coupling plural of modifiable bandage longitudinally.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a modifiable bandage system and method, constructed and operative according to the teachings of the present disclosure.

FIG. 1 is a bottom view of the modifiable bandage system during an ‘in-use’ condition, according to an embodiment of the disclosure.

FIG. 2 is a bottom view of the modifiable bandage system of FIG. 1, according to an embodiment of the present disclosure.

FIG. 3 is a top view of the modifiable bandage system of FIG. 1, according to an embodiment of the present disclosure.

FIG. 4 is a kit view of the modifiable bandage system of FIG. 1, according to an embodiment of the present disclosure.

FIG. 5 is a flow diagram illustrating a method of method of dressing a wound with a modifiable bandage system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a bandage and more particularly to a modifiable bandage system and method as used to improve the use of bandages for protection means.

Generally, the modifiable bandage system may be used to cover virtually all sizes of lacerations on a body. The modifiable bandages are similar to conventional bandages in that they adhere to a user's skin with an absorbent pad pressed against the wound. The modifiable bandage system however may be extended longitudinally and transversely by coupling multiple bandages together. The modifiable bandage system operates by using multiple bandages concurrently. Each individual bandage includes line markers on the back surface that instruct a user what portions of the bandage must be removed before coupling. A user may remove the indicated portion on at least one bandage and pair at least two adjacent absorbent pads together, effectively increasing the surface area covered by the absorbent pad material. This procedure of increasing the surface area may be performed as many times as needed to cover any size of wound.

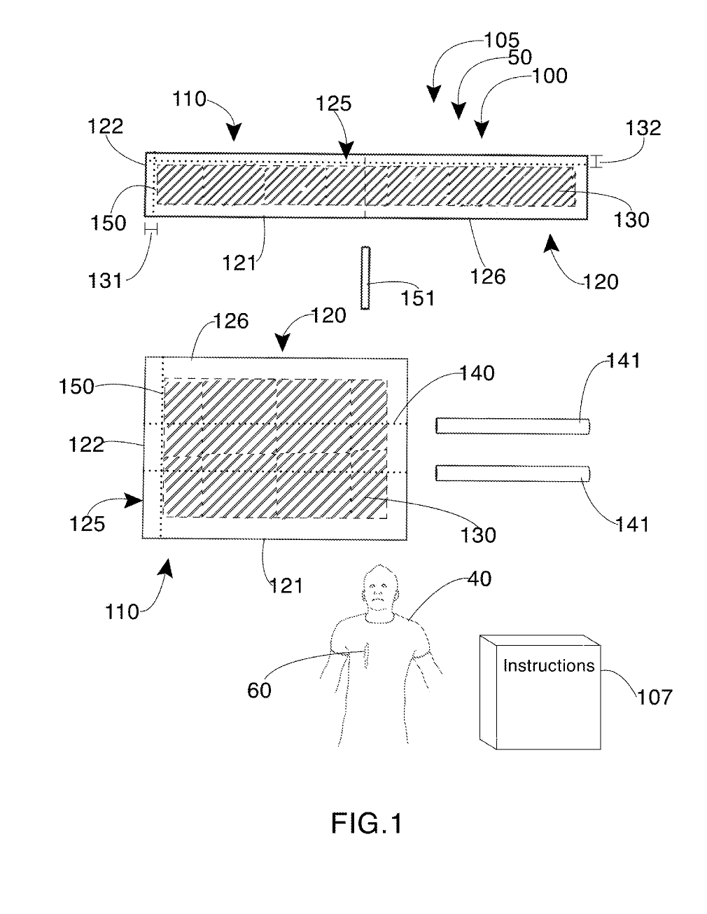

Referring now more specifically to the drawings by numerals of reference, there is shown in FIGS. 1-4, various views of a modifiable bandage system 100.

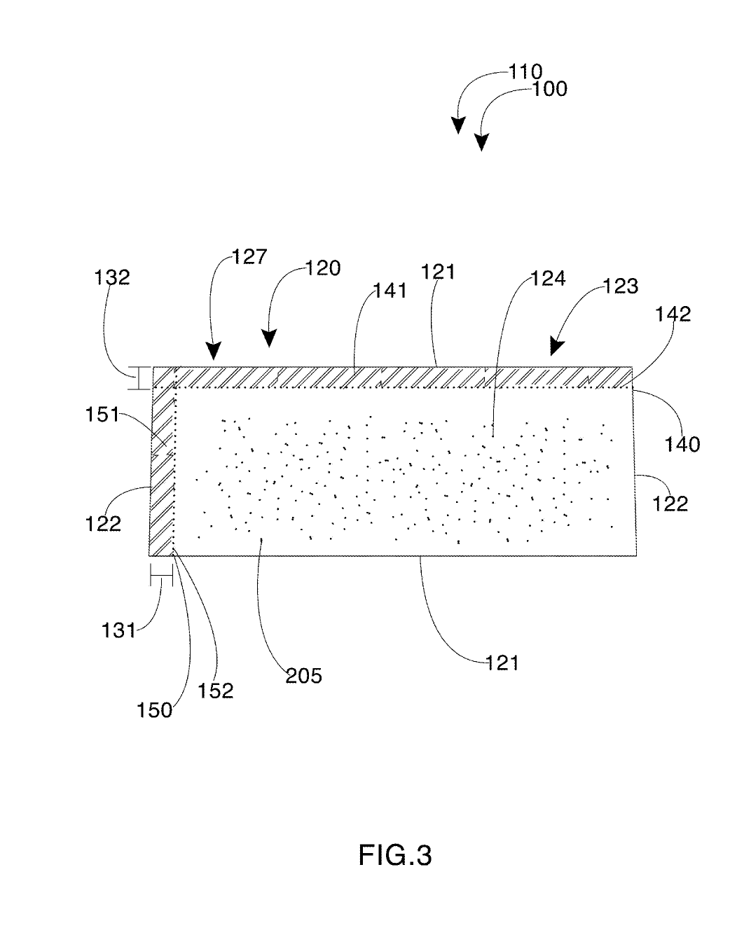

FIG. 1 shows a modifiable bandage system 100 during an ‘in-use’ condition 50, according to an embodiment of the present disclosure. Here, the modifiable bandage system 100 may be beneficial for use by a user 40 to create a larger bandage; certain embodiments are modular. As illustrated, the modifiable bandage system 100 may include a modifiable bandage 110. The modifiable bandage 110 includes a malleable material 120. The malleable material 120 is substantially rectangular in shape and has opposing long-edges 121 and opposing short-edges 122. The malleable material 120 has a top-surface 123 (FIG. 3) with a substantially fluid-proof laminate 124 (FIG. 3). The malleable material 120 has a bottom-surface 125 with an adhesive material 126. The modifiable bandage 110 further includes an absorbent material 130. The absorbent material 130 is disposed on the bottom-surface 125 of the malleable material 120. The absorbent material 130 extends longitudinally a first-distance 131 from each of the short-edges 122. The absorbent material 130 also extends transversely a second-distance 132 from each of the long-edges 121. The absorbent material 130 is oriented on the bottom-surface 125 of the malleable material 120 such that the absorbent material 130 is circumscribed by the adhesive material 126. A longitudinal severable indicia 140 is disposed on the top-surface 123 of the malleable material 120. The longitudinal severable indicia 140 extends between the opposing short-edges 122. The longitudinal severable indicia 140 is disposed the second-distance 132 from at least of the long-edges 121. The longitudinal severable indicia 140 defines a severable-longitudinal area 141 of the malleable material 120. A transverse severable indicia 150 is disposed on the top-surface 123 of the malleable material 120. The transverse severable indicia 150 extends between the opposing long-edges 121. The transverse severable indicia 150 is disposed the first-distance 131 from at least one of the short-edges 122. The transverse severable indicia 150 defines a severable-transverse area 151 of the malleable material 120.

As shown, the absorbent material 130 may be extended longitudinally by removal of the severable-transverse area 151 and coupling plural of the modifiable bandage 110. The absorbent material 130 may further be extended transversely by removal of the severable-longitudinal area 141 and coupling plural of the modifiable bandage 110. The modifiable bandage system 100 may be useful for covering various sizes of a wound 60. The modifiable bandage system 100 may be extended longitudinally and transversely to unlimited lengths allowing the modifiable bandage system 100 to be used for any sized wound 60 without the need for auxiliary materials.

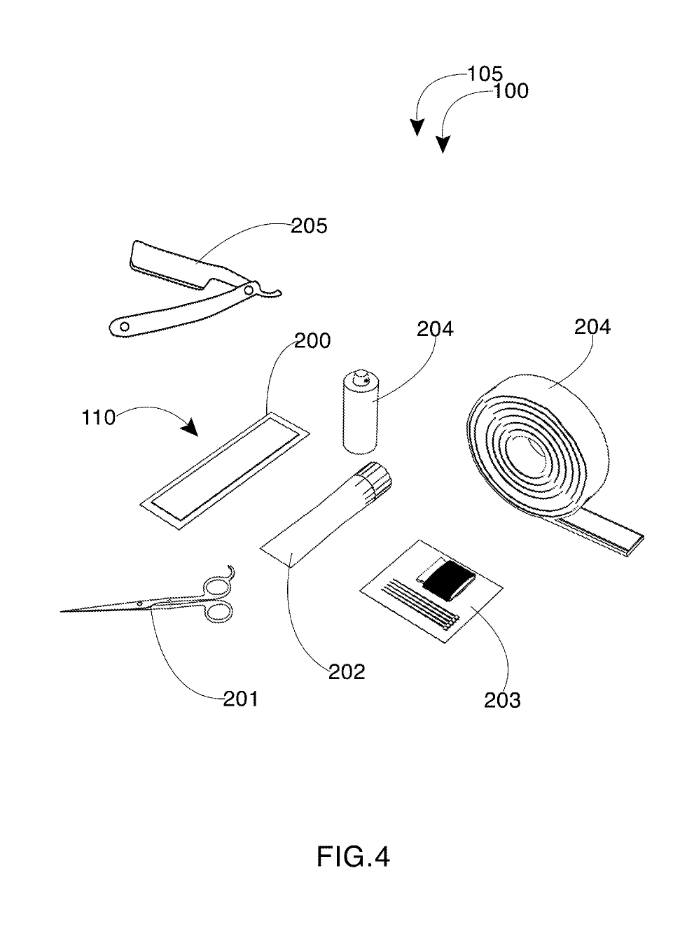

According to one embodiment, the modifiable bandage system 100 may be arranged as a kit 105, as shown in FIG. 4. In particular, the modifiable bandage system 100 may further include a set of instructions 107 and other implements corresponding to medical treatment. The instructions 107 may detail functional relationships in relation to the structure of the modifiable bandage system 100 such that the modifiable bandage system 100 can be used, maintained, or the like, in a preferred manner.

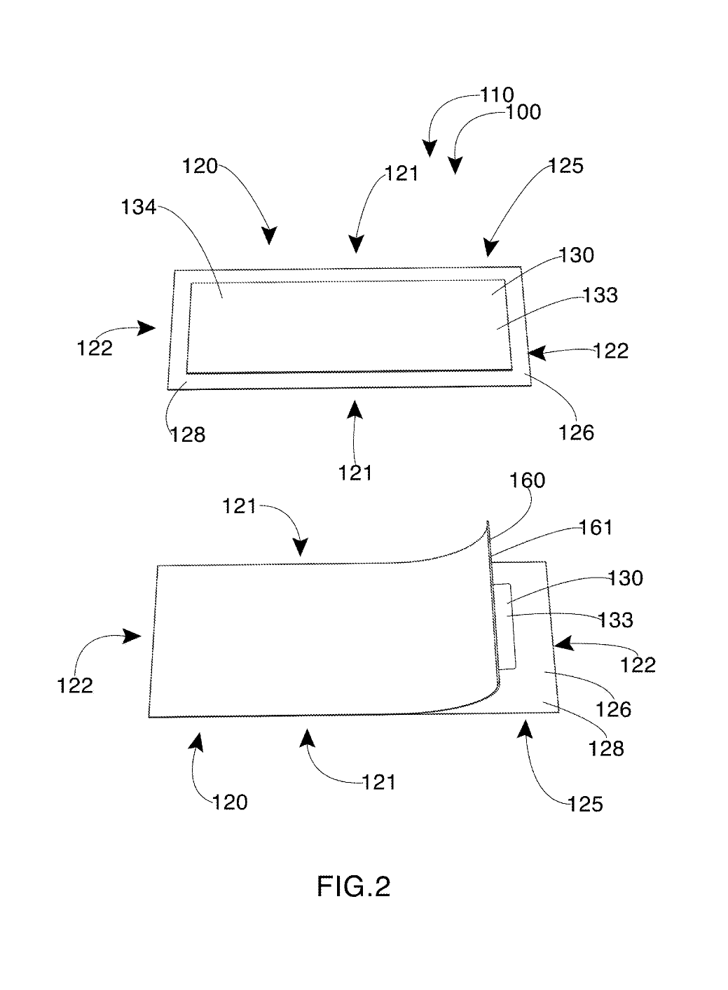

FIG. 2 shows the modifiable bandage system 100 of FIG. 1, according to an embodiment of the present disclosure. As above, the modifiable bandage system 100 may include a modifiable bandage 110 having comprising a malleable material 120. The malleable material 120 is substantially rectangular in shape and has opposing long-edges 121 and opposing short-edges 122. As shown, malleable material 120 has a bottom-surface 125 where disposed on the bottom-surface 125 is an absorbent material 130 circumscribed by an adhesive material 126. The absorbent material 130 may be made of cotton 134, and alternatively, a similar material. As the function of the modifiable bandage system 100 is to dress a wound 60 (FIG. 1), the adhesive material 126 may include an adhesive-integrated antiseptic 128. The adhesive-integrated antiseptic 128 may be useful for killing microbes that may surround the wound 60 that is being dressed. Killing the microbes that surround a wound 60 may prevent infection if the modifiable bandage 110 is displaced while attached a user 40 (FIG. 1). Similarly, the absorbent material 130 of the modifiable bandage 110 may include a material-integrated antiseptic 133. The material-integrated antiseptic 133 may be useful for killing microbes and keeping the wound 60 free from infection. Further pictured is a removable backing 160. The removable backing 160 removably attaches to the bottom-surface 125 of the malleable material 120. The removable backing 160 is designed to protect the adhesive material 126 from debris when the modifiable bandage 110 is not in use. The removable backing 160 may be made of wax paper 161, and alternatively, a similar material that allows for a user 40 (FIG. 1) to easily peel off the removable backing 160 from the adhesive material 126.

FIG. 3 is a top view of the modifiable bandage system 100 of FIG. 1, according to an embodiment of the present disclosure. The modifiable bandage 110 may include a malleable material 120 being substantially rectangular in shape. The malleable material 120 has opposing long-edges 121 and opposing short-edges 122. The malleable material 120 may comprise a cloth 127 having elastic properties. As shown, the malleable material 120 has a top-surface 123. The top-surface 123 includes a substantially fluid-proof laminate 124. The substantially fluid-proof laminate 124 may include a plurality of perforations 205. The plurality of perforations 205 may be used to allow the wound 60 (FIG. 1) to be exposed to air. Allowing the wound 60 to be exposed to air impedes the growth of bacteria. A longitudinal severable indicia 140 is disposed on the top-surface 123 of the malleable material 120. The longitudinal severable indicia 140 extends between the opposing short-edges 122, a second-distance 132 from at least one of the long-edges 121, defining a severable-longitudinal area 141. The longitudinal severable indicia 140 may include a longitudinal perforation pattern 142. The longitudinal perforation pattern 142 may be configured to aid with the separation of the severable-longitudinal area 141 from the modifiable bandage 110. A transverse severable indicia 150 is disposed on the top-surface 123 of the malleable material 120. The transverse severable indicia 150 extends between the opposing long-edges 121, a first-distance 131 from at least one of the short-edges 122, defining a severable-transverse area 151. The transverse severable indicia 150 may include a transverse perforation pattern 152. The transverse perforation pattern 152 may be configured to aid with the separation of the severable-transverse area 151 from the modifiable bandage 110. In a possible embodiment, the first-distance 131 and the second-distance 132 may be equal in length.

FIG. 4 is a kit view of the modifiable bandage system 100 of FIG. 1, according to an embodiment of the present disclosure. The present figure illustrates the various implements that may be included in the modifiable bandage system 100 when arranged as a kit 105. The modifiable bandage system 100 may comprise a packaging 200. The packaging 200 configured to envelope the modifiable bandage 110. The packaging 200 may be used to store the modifiable bandage 110 until use. The modifiable bandage system 100 may include a pair of scissors 201. The pair of scissors 201 may be configured to sever the severable-longitudinal area 141 (FIG. 1) and, the severable-transverse area 151 (FIG. 1). A container of antiseptic solution 202 may be included with the modifiable bandage system 100. The container of antiseptic solution 202 may be useful for fighting infection. A sewing kit 203 may be included with the modifiable bandage system 100. The sewing kit 203 may be configured to stitch a wound 60 (FIG. 1). An occlusive dressing 204 may be included with the modifiable bandage system 100. The occlusive dressing 204 is used for sealing particular types of a wound 60 and their surrounding tissue off from air, fluids, and harmful contaminants. A razor 205 may be included for removing hair that circumscribes a wound 60.

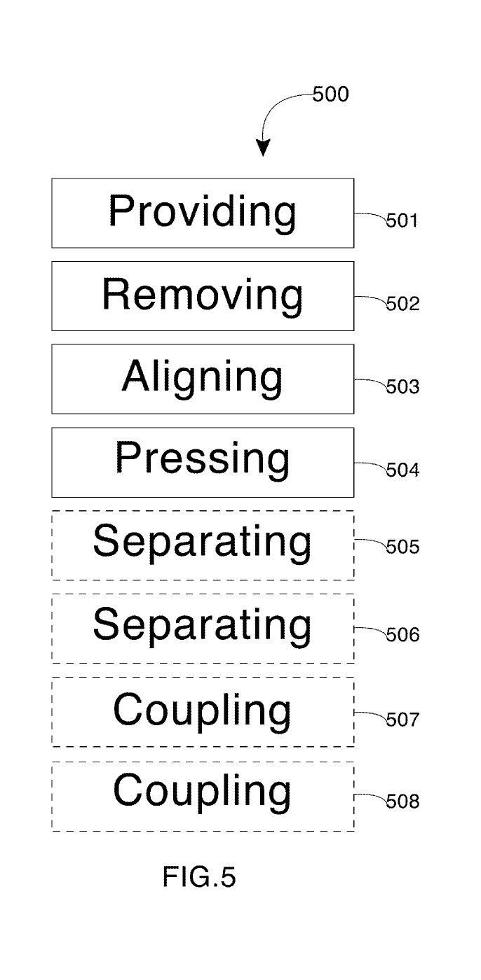

FIG. 5 is a flow diagram illustrating a method for method of dressing a wound with a modifiable bandage system 500, according to an embodiment of the present disclosure. In particular, the method for method of dressing a wound with a modifiable bandage system 500 may include one or more components or features of the modifiable bandage system 100 as described above. As illustrated, the method for method of dressing a wound with a modifiable bandage system 500 may include the steps of: step one 501, providing a modifiable bandage system, the modifiable bandage system comprising a modifiable bandage, the modifiable bandage including: a malleable material, the malleable material substantially rectangular in shape having opposing long-edges and opposing short-edges, the malleable material having a top-surface wherein a substantially fluid-proof laminate is disposed on the top-surface and, a bottom-surface wherein an adhesive material is disposed on the bottom-surface; an absorbent material, the absorbent material is disposed on the bottom-surface of the malleable material, the absorbent material extending longitudinally a first-distance from each of the short-edges, and transversely a second-distance from each of the long-edges, wherein the absorbent material is circumscribed by the adhesive material; a longitudinal severable indicia, the longitudinal severable indicia disposed on the top-surface of the malleable material, the longitudinal severable indicia extending between opposing the short-edges the second-distance from at least one of the long-edges defining a severable-longitudinal area; a transverse severable indicia, the transverse severable indicia disposed on the top-surface of the malleable material, the transverse severable indicia extending between opposing long-edges the first-distance from at least one of the short-edges defining a severable-transverse area; a removable backing, the removable backing removably attached to the bottom-surface of the malleable material; wherein the absorbent material may be extended longitudinally by removal of the severable-transverse area and coupling plural of the modifiable bandage; wherein the absorbent material may be extended transversely by removal of the severable-longitudinal area and coupling plural of the modifiable bandage; step two 502, removing the removable backing from the bottom-surface of the malleable material; step three 503, aligning the absorbent material over the wound; step four 504, pressing the modifiable bandage onto the wound such that the adhesive material circumscribes the wound and absorbent material covers the wound; step five 505, separating the severable-longitudinal area from the modifiable bandage; step six 506, separating the severable-transverse area from the modifiable bandage; step seven 507, coupling plural of modifiable bandage longitudinally; step eight 508, coupling plural of modifiable bandage transversely;

It should be noted that steps 505, 506, 507 and, 508 are optional steps and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for of dressing a wound with a modifiable bandage system 100 are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in said appended claims:

1. A modifiable bandage system, said modifiable bandage system comprising:
a modifiable bandage, said modifiable bandage including:
a malleable material, said malleable material substantially rectangular in shape having opposing long-edges and opposing short-edges, said malleable material having a top-surface wherein a substantially fluid-proof laminate is disposed on said top-surface and, a bottom-surface wherein an adhesive material is disposed on said bottom-surface;
an absorbent material, said absorbent material is disposed on said bottom-surface of said malleable material, said absorbent material extending longitudinally a first-distance from each of said short-edges, and transversely a second-distance from each of said long-edges, wherein said absorbent material is circumscribed by said adhesive material;
a longitudinal severable indicia, said longitudinal severable indicia disposed on said top-surface of said malleable material, said longitudinal severable indicia extending between said opposing short-edges said second-distance from at least one of said long-edges defining a severable-longitudinal area;
a transverse severable indicia, said transverse severable indicia disposed on said top-surface of said malleable material, said transverse severable indicia extending between said opposing long-edges said first-distance from at least one of said short-edges defining a severable-transverse area;
a removable backing, said removable backing removably attached to said bottom-surface of said malleable material;
wherein said absorbent material may be extended longitudinally by removal of said severable-transverse area and coupling plural of said modifiable bandage; and,
wherein said absorbent material may be extended transversely by removal of said severable-longitudinal area and coupling plural of said modifiable bandage;

2. The modifiable bandage system of claim 1, wherein said modifiable bandage system further comprises a packaging, said packaging configured to envelope said modifiable bandage.

3. The modifiable bandage system of claim 1, wherein said modifiable bandage system further comprises a pair of scissors, said pair of scissors configured to sever said severable-longitudinal area and, said severable-transverse area.

4. The modifiable bandage system of claim 1, wherein said modifiable bandage system further comprises a container of antiseptic solution, said container of antiseptic solution configured to treat a wound.

5. The modifiable bandage system of claim 1, wherein said modifiable bandage system further comprises a sewing kit, said sewing kit configured to stitch said wound.

6. The modifiable bandage system of claim 1, wherein said modifiable bandage system further comprises an occlusive dressing.

7. The modifiable bandage system of claim 1, wherein said malleable material comprises a cloth, said cloth having elastic properties.

8. The modifiable bandage system of claim 1, wherein said substantially fluid-proof laminate includes a plurality of perforations.

9. The modifiable bandage system of claim 1, wherein said adhesive material includes an adhesive-integrated antiseptic.

10. The modifiable bandage system of claim 1, wherein said absorbent material includes a material-integrated antiseptic.

11. The modifiable bandage system of claim 1, wherein said first-distance and said second-distance are equal in length.

12. The modifiable bandage system of claim 1, wherein said longitudinal severable indicia includes a longitudinal perforation pattern, said longitudinal perforation pattern configured to aid in separation of said severable-longitudinal area from said modifiable bandage.

13. The modifiable bandage system of claim 1, wherein said transverse severable indicia includes a transverse perforation pattern, said transverse perforation pattern configured to aid in separation of said severable-transverse area from said modifiable bandage.

14. The modifiable bandage system of claim 1, wherein said removable backing comprises wax paper.

15. The modifiable bandage system of claim 1, wherein said modifiable bandage system further includes a razor configured to remove hair circumscribing said wound.

16. The modifiable bandage system of claim 1, wherein said absorbent material is cotton.

17. A modifiable bandage system, said modifiable bandage system comprising:
a modifiable bandage, said modifiable bandage including:
a malleable material, said malleable material substantially rectangular in shape having opposing long-edges and opposing short-edges, said malleable material having a top-surface wherein a substantially fluid-proof laminate is disposed on said top-surface and, a bottom-surface wherein an adhesive material is disposed on said bottom-surface,
an absorbent material, said absorbent material is disposed on said bottom-surface of said malleable material, said absorbent material extending longitudinally a first-distance from each of said short-edges, and transversely a second-distance from each of said long-edges, wherein said absorbent material is circumscribed by said adhesive material,
a longitudinal severable indicia, said longitudinal severable indicia disposed on said top-surface of said malleable material, said longitudinal severable indicia extending between opposing said short-edges said second-distance from at least one of said long-edges defining a severable-longitudinal area,
a transverse severable indicia, said transverse severable indicia disposed on said top-surface of said malleable material, said transverse severable indicia extending between opposing long-edges said first-distance from at least one of said short-edges defining a severable-transverse area,
a removable backing, said removable backing removably attached to said bottom-surface of said malleable material,
wherein said absorbent material may be extended longitudinally by removal of said severable-transverse area and coupling plural of said modifiable bandage, and,
wherein said absorbent material may be extended transversely by removal of said severable-longitudinal area and coupling plural of said modifiable bandage,
wherein said modifiable bandage system further comprises a packaging, said packaging configured to envelope said modifiable bandage;
wherein said modifiable bandage system further comprises a pair of scissors, said pair of scissors configured to sever said severable-longitudinal area and, said severable-transverse area;
wherein said modifiable bandage system further comprises a container of antiseptic solution, said container of antiseptic solution configured to treat a wound;
wherein said modifiable bandage system further comprises a sewing kit, said sewing kit configured to stitch said wound;
wherein said modifiable bandage system further comprises an occlusive dressing;
wherein said malleable material comprises a cloth, said cloth having elastic properties;
wherein said substantially fluid-proof laminate includes a plurality of perforations;
wherein said adhesive material includes an adhesive-integrated antiseptic;
wherein said absorbent material includes a material-integrated antiseptic;
wherein said first-distance and said second-distance are equal in length;
wherein said longitudinal severable indicia includes a longitudinal perforation pattern, said longitudinal perforation pattern configured to aid in separation of said severable-longitudinal area from said modifiable bandage;
wherein said transverse severable indicia includes a transverse perforation pattern, said transverse perforation pattern configured to aid in separation of said severable-transverse area from said modifiable bandage;
wherein said removable backing comprises wax paper;
wherein said modifiable bandage system further includes a razor configured to remove hair circumscribing said wound;
wherein said absorbent material is cotton;

18. The modifiable bandage system of claim 17, further comprising set of instructions; and
wherein said modifiable bandage system is arranged as a kit.

19. A method of dressing a wound with a modifiable bandage system, said method comprising said steps of:
providing a modifiable bandage system, said modifiable bandage system comprising a modifiable bandage, said modifiable bandage including: a malleable material, said malleable material substantially rectangular in shape having opposing long-edges and opposing short-edges, said malleable material having a top-surface wherein a substantially fluid-proof laminate is disposed on said top-surface and, a bottom-surface wherein an adhesive material is disposed on said bottom-surface; an absorbent material, said absorbent material is disposed on said bottom-surface of said malleable material, said absorbent material extending longitudinally a first-distance from each of said short-edges, and transversely a second-distance from each of said long-edges, wherein said absorbent material is circumscribed by said adhesive material; a longitudinal severable indicia, said longitudinal severable indicia disposed on said top-surface of said malleable material, said longitudinal severable indicia extending between opposing said short-edges said second-distance from at least one of said long-edges defining a severable-longitudinal area; a transverse severable indicia, said transverse severable indicia disposed on said top-surface of said malleable material, said transverse severable indicia extending between opposing long-edges said first-distance from at least one of said short-edges defining a severable-transverse area; a removable backing, said removable backing removably attached to said bottom-surface of said malleable material; wherein said absorbent material may be extended longitudinally by removal of said severable-transverse area and coupling plural of said modifiable bandage; wherein said absorbent material may be extended transversely by removal of said severable-longitudinal area and coupling plural of said modifiable bandage;
removing said removable backing from said bottom-surface of said malleable material;
aligning said absorbent material over said wound;
pressing said modifiable bandage onto said wound such that said adhesive material circumscribes said wound and absorbent material covers said wound;

20. The method of claim 19, further comprising said steps of separating said severable-longitudinal area from said modifiable bandage;
separating said severable-transverse area from said modifiable bandage;
coupling plural of modifiable bandage longitudinally;
coupling plural of modifiable bandage transversely;
